(19) United States
(12) Patent Application Publication
(10) Pub. No.: US 2026/0091043 A1
(43) Pub. Date: Apr. 2, 2026

(54) COMPOSITIONS AND METHODS FOR TREATING MYCOBACTERIUM INFECTIONS

(71) Applicant: DATA2DISCOVERY, INC., Bloomington, IN (US)

(72) Inventors: John David WILD, Bloomington, IN (US); Jeremy Joseph YANG, Albuquerque, NM (US); Sean EKINS, Fuquay Varina, NC (US)

(21) Appl. No.: 19/113,756

(22) PCT Filed: Sep. 20, 2023
(86) PCT No.: PCT/US2023/033252

§ 371 Date: Mar. 20, 2025

Related U.S. Application Data
(60) Provisional application No. 63/408,677, filed on Sep. 21, 2022.

(51) Int. Cl. A61K 31/609; A61P 31/04; A61P 31/06

(52) U.S. Cl. CPC A61K 31/609; A61P 31/04; A61P 31/06

(57) ABSTRACT

Described herein are treatments of infections and diseases caused by pathogenic bacteria, including the treatment of infections and diseases caused by Mycobactertaceae.

### TECHNICAL FIELD

The invention described herein pertains to the treatment of infections and diseases caused by pathogenic bacteria and related species. The invention described herein also pertains to the treatment of infections and diseases caused by Mycobacteriaceae. The invention described herein also pertains to the treatment of infections and diseases caused by Mycobacterium genus.

### BACKGROUND AND SUMMARY OF THE INVENTION

Mycobacteriaceae is a family of Actinobacteria, and includes the genus Mycobacterium. Mycobacterium genus infections are responsible for a large number of serious diseases, including tuberculosis, leprosy, and many others. In addition, Mycobacterium infections are responsible for complicating and exacerbating other diseases, such as cystic fibrosis. Mycobacterium infections are also responsible for high mortality rates in immunocompromised patients, such as in the elderly and in patients afflicted with HIV and AIDS.

Tuberculosis (TB) is a common and often deadly infectious disease caused by Mycobacterium. In humans, TB is caused mainly by Mycobacterium tuberculosis. An estimated one-third of the world's current population has been infected with M. tuberculosis, and reportedly, new infections occur at a rate of one per second. Though the proportion of people in the general population who become sick with TB each year is relatively stable, because of population growth, the actual number of new cases is still increasing. Moreover, the negative impact of the disease is compounded by the emergence of resistant strains of TB. Further confounding treatment efforts, many of the infected are asymptomatic, having so-called latent TB, but reportedly, are still able to infect others.

Mycobacterium abscessus complex (MABSC) is a group of nontuberculous mycobacteria (NTM) species that are common soil and water contaminants, and includes M. abscessus, M. bolletii, or M. massiliense. MABSC is rapidly growing, and often multidrug-resistant. MABSC most commonly causes chronic lung infection and skin and soft tissue infection (SSTI); however, MABSC can reportedly cause infection in almost any human organ, and is notably dangerous to immunocompromised patients with suppressed immune systems. For example, M. abscessus can cause lung disease, skin infections, central nervous system infections, bacteremia, eye infections, and other diseases. M. abscessus can cause skin infections in immunodeficient patients, patients who have recently undergone surgery, individuals after tattooing, acupuncture, or exposure to hot springs or spas. M. abscessus can be associated with middle-ear infections (otitis media). Chronic lung disease occurs most commonly in hosts with underlying lung disease, such as cystic fibrosis, chronic obstructive pulmonary disease (COPD), bronchiectasis, and/or prior tuberculosis.

Mycobacterium avium complex (MAC) is a group of genetically related bacteria belonging to the genus Mycobacterium. MAC includes, for example, Mycobacterium avium subspecies avium (MAA), Mycobacterium avium subspecies hominis (MAH), and Mycobacterium avium subspecies paratuberculosis (MAP). MAC infection is a serious health concern for immunocompromised patients, including the elderly, and such infections are sometimes termed Lady Windermere syndrome. MAC infection is also a serious health concern for the estimated 38 million people world-wide having with AIDS. MAC is the causative agent for more than 90% of the non-tuberculous Mycobacterium infections in those patients. Typical treatment regimens require combination therapy, and consist of clarithromycin, ethambutol, and a rifamycin; however. treatment failures have been reported. MAC can also cause certain forms of hypersensitivity pneumonitis (HP), also known as Hot Tub Lung.

Mycobacterium genus also causes leprosy, also known as Hansen's disease, which is a chronic infectious disease that damages the peripheral nerves and targets the skin, eyes, nose, and muscles. Leprosy can occur at all ages from infancy to elderly. Leprosy is most often caused by M. Leprae, but M. lepraemurium and M. lepromatosis infections are also reported.

It is well known that Mycobacterium infections are difficult to treat. Mycobacterium genus are neither truly Gram negative nor Gram positive. Mycobacterium genus are hardy due to their cell wall, making them naturally resistant to a most antibiotics that disrupt cell-wall biosynthesis, such as penicillin. In addition, their unique cell wall allows them to survive long exposure to acids, alkalis, detergents, oxidative bursts, lysis by complement, and many other antibiotics. Mycobacterium genus are generally susceptible only to the macrolide antibiotics, such as clarithromycin, and ansamycins, such as rifamycin. Any given compound's antibiotic activity against other bacteria is generally a poor predictor of activity against Mycobacterium genus.

New compounds, compositions, and methods are needed to treat Mycobacterium genus infections.

It has been surprisingly discovered that salicylanilides are therapeutically effective agents against Mycobacterium genus, including, but not limited to M. tuberculosis and M. abscessus.

Described herein are compounds, compositions, medicaments, formulations, kits, and methods for treating a host animal with Mycobacterium genus infections, including MTBC, MABC, MAC, and M. leprae infections, and diseases caused by any of the foregoing. The compositions, formulations, and methods include a therapeutically effective amount of one or more salicylanilides, prodrugs thereof, or salts of any of the foregoing.

In one illustrative embodiment of the invention, compounds, compositions, medicaments, formulations, kits, and methods are described herein for treating a host animal with TB, or having a MTBC or M. tuberculosis infection. In another illustrative embodiment, compounds, compositions, medicaments, formulations, kits, and methods are described herein for treating a host animal with dormant forms of tuberculosis, including latent TB. In another illustrative embodiment, compounds, compositions, medicaments, formulations, kits, and methods are described herein for treating TB caused at least in part by resistant organisms, including macrolide resistant TB, multidrug-resistant tuberculosis (MDR-TB), and extensively drug-resistant TB (XDR TB).

In another illustrative embodiment, compounds, compositions, medicaments, formulations, kits, and methods are described herein for treating a host animal having a MABC infection, such as a M. abscessus, M. bolletii, or M. massiliense infection.

In another illustrative embodiment, compounds, compositions, medicaments, formulations, kits, and methods are described herein for treating a host animal having a MAC infection, such as a M. avium or M. avium para tuberculosis infection. In another illustrative embodiment, compounds, compositions, medicaments, formulations, kits, and methods are described herein for treating an immunocompromised host animal, such as a host animal with HIV or AIDS, having a MAC infection, such as a M. avium or M. avium para tuberculosis infection. In another illustrative embodiment, compounds, compositions, medicaments, formulations, kits, and methods are described herein for treating a host animal having a MAC infection, such as a M. avium or M. avium para tuberculosis infection caused at least in part by a macrolide resistant organism, including multidrug resistant MAC.

In another illustrative embodiment, compounds, compositions, medicaments, formulations, kits, and methods are described herein for treating a host animal with leprosy, or having a M. Leprae, M. lepraemurium, or M. lepromatosis infection.

### BRIEF DESCRIPTION OF THE DRAWINGS

FIG. 1 shows inhibition of M. tuberculosis by compounds described herein.

FIG. 2 shows inhibition of M. abscessus by compounds described herein.

### DETAILED DESCRIPTION

Several illustrative embodiments of the invention are described by the following delineated clauses:

A composition comprising a salicylanilide or a prodrug thereof, or a hydrate or solvate of any of the foregoing, or a salt of the foregoing, for use in treating an infection caused at least in part by pathogenic bacteria.

The composition of the preceding clause wherein the bacteria include Mycobacteriaceae.

The composition of any one of the preceding clauses wherein the bacteria include Mycobacterium tuberculosis complex (MTBC), such as M. tuberculosis, Mycobacterium abscessus complex (MABC), such as M. abscessus, M. bolletii, or M. massiliense, Mycobacterium avium complex (MAC), such as M. avium or M. avium para tuberculosis, Mycobacteria causing leprosy, such as M. Leprae, M. lepraemurium, or M. lepromatosis, Mycobacteria comorbid with cystic fibrosis, such as M. chitae or M. fallax, or other Mycobacteria such as M. ulcerans, or any combination of the foregoing.

The composition of any one of the preceding clauses wherein the bacteria include Mycobacterium tuberculosis complex (MTBC), such as M. tuberculosis.

The composition of any one of the preceding clauses wherein the bacteria include Mycobacterium abscessus complex (MABC), such as M. abscessus, M. bolletii, or M. massiliense, or a combination thereof.

The composition of any one of the preceding clauses wherein the bacteria include Mycobacterium avium complex (MAC), such as M. avium or M. avium para tuberculosis, or a combination thereof.

The composition of any one of the preceding clauses wherein the bacteria include Mycobacteria causing leprosy, such as M. Leprae, M. lepraemurium, or M. lepromatosis, or a combination thereof.

The composition of any one of the preceding clauses wherein the bacteria include Mycobacteria comorbid with cystic fibrosis, such as M. chitae or M. fallax, or a combination thereof.

The composition of any one of the preceding clauses wherein the composition is configured for oral administration.

The composition of any one of the preceding clauses wherein the composition is configured for administration by inhalation.

The composition of any one of the preceding clauses wherein the host animal is a mammal, such as livestock, a companion animal, or a human.

The composition of any one of the preceding clauses wherein the host animal is immunocompromised.

The composition of any one of the preceding clauses wherein the composition is configured for administration to an immunocompromised host animal.

The composition of any one of the preceding clauses wherein the salicylanilide is selected from the formula

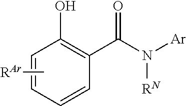

and prodrugs thereof; and salts of any of the foregoing; where
R^{Ar} represents one or more aryl substituents;
R^{N} is H, alkyl, or acyl; and
Ar is an optionally substituted aryl group or heteroaryl group.

The composition of any one of the preceding clauses wherein R^{Ar} is one or more halo groups, hydroxy, amino, or heteroaryl, such as pyrrol-1-yl, or any combination of the foregoing.

The composition of any one of the preceding clauses wherein Ar is phenyl or substituted phenyl, including phenyl substituted with one or more halo groups, hydroxy, amino, nitro, alkyl, haloalkyl, including trifluoromethyl, alkoxy, including trifluoromethoxy, alkenyl, alkynyl, optionally substituted arylalkyl, optionally substituted aryloxy, or (optionally substituted phenyl)(cyano)methyl, or any combination of the foregoing.

The composition of any one of the preceding clauses wherein R^{N} is H.

The composition of any one of the preceding clauses wherein the compound is selected from the formula

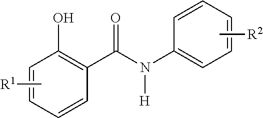

where R¹ is 5-Cl and R² is 4-Cl, R¹ is 5-Cl and R² is c-Cl, or R¹ is 5-Cl and R² is 4-Br.

The composition of any one of the preceding clauses wherein the composition comprises salicylanilide, niclosamide, oxyclozanide, rafoxanide, closantel. 5-chlorosalicylanilide (CSA), 4′-bromosalicylanilide (BSA), 5-bromo-4′-chlorosalicylanilide (BCSA), 3,3′4′,5-tetrachlorosalicylanilide (TCSA), bromochlorosalicylanilide, or a combination thereof.

The composition of any one of the preceding clauses wherein the composition comprises 5-bromo-N-(3,5-dichlorophenyl)-2-hydroxy-benzamide, 5-chloro-N-(2,4-difluorophenyl)-2-hydroxy-benzamide, 3,5-dichloro-N-(2,4-difluorophenyl)-4-pyrrol-1-yl-benzamide, 4-amino-3,5-dichloro-N-(3,5-dichlorophenyl)benzamide, 5-chloro-N-(2-chloro-4-nitro-phenyl)-2-hydroxy-benzamide, clioxanide, or a combination thereof.

The composition of any one of the preceding clauses wherein the composition comprises dibromsalan (DBS), metabromsalan (MBS), tribromsalan (TBS. 3,5,4′-tribromosalicylanilide), resorantel, 4′,5-dibromosalicylanilide or a combination thereof.

The composition of any one of the preceding clauses wherein the prodrug is an 2-O-acyl derivative, such as 2-O-acetyl.

A pharmaceutical composition comprising a compound or composition of any of the clauses recited herein, and optionally comprising one or more carriers, diluents, excipients, and the like, and combinations thereof.

A unit dose comprising a compound or composition of any of the clauses recited herein, where the unit dose is in single or divided form, and includes a therapeutically effective amount of the compound or composition for treating a host animal with a bacterial infection, as described herein. The unit doses includes a therapeutically effective amount of the one or more compounds for treating a Mycobacterium genus infection in a host animal. The unit doses are in single or divided form, and may correspond to a daily dosage amount, or adjusted to a periodic amount that is shorter, including for multiple daily doses, or longer, including weekly or monthly doses. It is to be understood that the compositions may include other components and/or ingredients, including, but not limited to, other therapeutically active compounds, and/or one or more carriers, vehicles, diluents, adjuvants, excipients, and the like, and combinations thereof.

A method for treating a bacterial infection in a host animal, as described herein, the method comprising administering a therapeutically effective amount of a compound or composition of any of the clauses recited herein, or a unit dose comprising any of the foregoing, in single or divided form, to the host animal.

Use of a compound or composition of any of the clauses recited herein in the manufacture of a medicament, including a unit dose, in single or divided form, for treating a bacterial infection in a host animal, as described herein.

In each of the foregoing and each of the following embodiments, unless otherwise indicated, it is also to be understood that the formulae include and represent any and all crystalline forms, partially crystalline forms, and non-crystalline and/or amorphous forms of the compounds, including partially ordered forms, disordered forms, liquid crystal forms, and meso phases of any of the foregoing.

In each of the foregoing and each of the following embodiments, unless otherwise indicated, it is also to be understood that the formulae include and represent each possible isomer, such as stereoisomers and geometric isomers, both individually and in any and all possible mixtures.

In each of the foregoing and each of the following embodiments, unless otherwise indicated, it is also to be understood that the transitional phrase “consisting essentially of” means that the scope of the corresponding composition, unit dose, method or use is understood to encompass the specified compounds or recited steps, and those that do not materially affect the basic and novel characteristics of the invention described herein. For example, a method described herein that consists essentially of a single compound, or genus of compounds, is understood to represent a monotherapy for the recited disease. Though the monotherapy may include co-administration of one or more carriers, vehicles, diluents, adjuvants, excipients, and the like, and combinations thereof, and/or include co-administration of one or more additional active pharmaceutical ingredients, those latter additional active pharmaceutical ingredients are to be understood to be for treating diseases and/or symptoms distinct from treating the underlying conditions described herein.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds, or spatial arrangements, such as cis, trans, syn, and anti, relative configurations on a ring. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₆, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like Illustratively, such particularly limited length alkenyl and/or alkynyl groups, including C₂-C₈, C₂-C₆, and C₂-C₄, and C₃-C₈, C₃-C₆, and C₃-C₄, and the like may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. It is to be further understood that in certain embodiments alkenylene and/or alkynylene may each be advantageously of limited length, including C₂-C₂₄, C₂-C₁, C₂-C₆, C₂-C₆, and C₂-C₄, and C₃-C₂₄, C₃-C₁₂, C₃-C₈, C₃-C₆, and C₃-C₄, and the like. Illustratively, such particularly limited length alkenylene and/or alkynylene groups, including C₂-C₈, C₂-C₆, and C₂-C₄, and C₃-C₆, C₃-C₆, and C₃-C₄, and the like may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarvlalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arvlalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarvlalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arvlalkenylsulfonyl, arylalkynylsulfonyl, heteroarylsulfonyl, heteroarvlalkylsulfonyl, heteroarylalkenylsulfonyl, heteroarylalkynylsulfonyl, acylsulfonyl, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups, also referred to herein as aryl substituents or heteroaryl substituents, respectively, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165. It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “solvates” refers to compounds described herein complexed with a solvent molecule. It is appreciated that compounds described herein may form such complexes with solvents by simply mixing the compounds with a solvent, or dissolving the compounds in a solvent. It is appreciated that where the compounds are to be used as pharmaceuticals, such solvents are pharmaceutically acceptable solvents. It is further appreciated that where the compounds are to be used as pharmaceuticals, the relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In some embodiments, the solvates are amorphous, and in other embodiments, the solvates are crystalline.

As used herein, the term “composition” generally refers to any product comprising the specified ingredients in the specified amounts, as well as any product which results, directly or indirectly, from combinations of the specified ingredients in the specified amounts. It is appreciated that certain functional groups, such as the hydroxy, amino, and like groups form complexes and/or coordination compounds with water and/or various solvents, in the various physical forms of the compounds. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. In addition, it is to be understood that the compositions may be prepared from various co-crystals of the compounds described herein. Accordingly, such pharmaceutical compositions that recite compounds described herein are to be understood to include each of, or any combination of, the various morphological forms and/or solvate or hydrate forms of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2006)).

As used herein, the term “host animal” generally refers to mammals, including humans, companion animals, and livestock animals. A host animal in need is a host animal infected with or exposed to one or more infectious pathogens, such as Mycobacteriaceae. An immunocompromised host animal is one that is immunodeficient wherein the host animal immune system's ability to fight infectious disease is compromised or entirely absent. Most cases of immunodeficiency are acquired, secondary immunodeficiency, such as those patients with HIV or taking immunosuppressing drugs, but some host animals are born with defects in the immune system, primary immunodeficiency. Transplant host animals take medications to suppress their immune system as an anti-rejection measure, as do some host animals suffering from an over-active immune system or an autoimmune disease. An immunocompromised host animal may be particularly vulnerable to opportunistic infections, in addition to normal infections that in a host animal that is not immunocompromised might not otherwise have any adverse effects.

As used herein, the term “inhibiting” generally includes its generally accepted meaning which includes prohibiting, preventing, restraining, slowing, stopping, and/or reversing progression, severity of the infection and/or any resultant symptom of the infection. As such, the methods described herein include both clinical therapeutic and/or prophylactic administration, as appropriate.

As used herein, the term “therapeutically effective amount” generally refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed: the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

As used herein, the term “carrier” generally refers to any ingredient other than the active components in a formulation. The choice of carrier will to a large extent depend on factors such as the particular mode of administration, the effect of the carrier on solubility and stability, and the nature of the dosage form.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Illustrative formats for oral administration include, but are not limited to, tablets, capsules, elixirs, syrups, and the like.

Illustrative formats for administration by inhalation include, but are not limited to, nasal sprays and aerosols, inhalable powders, and the like.

The effective use of the compounds, compositions, and methods described herein for treating or ameliorating one or more effects of a bacterial infection using one or more compounds described herein may be based upon animal models, such as murine, canine, porcine, and non-human primate animal models of disease. For example, it is understood that bacterial infection in humans may be characterized by a loss of function, and/or the development of symptoms, each of which may be elicited in animals, such as mice, and other surrogate test animals.

In addition, it is to be understood that therapeutically effect doses administered in animal models may be used to calculate corresponding therapeutically effect doses for administration to other host animals, including humans. Illustrative corresponding doses may be calculated using the “Guidance for Industry Estimating the Maximum Safe Starting Dose in Initial Clinical Trials for Therapeutics in Adult Healthy Volunteers” published by FDA, and found at http://www.fda.gov/media/72309/download, and which is incorporated herein in its entirety by reference.

The following examples further illustrate specific embodiments of the invention; however, the following illustrative examples should not be interpreted in any way to limit the invention. Unless otherwise indicated, all starting compounds, reagents, and solvents used in the following examples are available from commercial suppliers.

### EXAMPLES

METHOD EXAMPLE. Resazurin Microtitre Assay (REMA). Test compounds are prepared as DMSO solutions. Kanamycin, a positive control, is prepared in deionized water and sterile filtered. Using non-treated polystyrene 96-well plates (e.g., Corning), test compounds are serially two-fold diluted in triplicate in 7H9 broth (Difco) supplemented with albumin dextrose saline (ADS; 10 g/L bovine serum albumin fraction V, 4 g/L dextrose, 1.6 g/L NaCl), 0.5% glycerol, and 0.1% Tyloxapol (7AGT). Test Mycobacterium, such as M abscessus strain ATCC 19977 (smooth), M. tuberculosis strain H37Rv, and the like, are grown in 7AGT until mid-logarithmic growth is reached. Cells are passed through a 40 μM cell strainer and allowed to settle. Culture cell density is measured by optical density (OD₆₀₀) and diluted to reach a final density of about 1×10⁵ cells/well. All wells, including test compounds and kanamycin controls, contain a final concentration of 1% DMSO and 200 μL total volume. The kanamycin control is diluted in 2-fold steps ranging from 0.2-82.5 μM. Plates are incubated for 48 hrs at 37° C., 100 rpm before adding 20 μL resazurin solution (125 μg/mL in phosphate buffered saline). Following the addition of resazurin, plates are incubated in the dark for an additional 24 hrs. Fluorescence is measured with an excitation at 544 nm and emission at 590 nm, with for example a Molecular Devices. SpectraMax M2 microplate reader (California, USA).

METHOD EXAMPLE. M. tuberculosis (H37RV strain) assay. Frozen stocked H37RV is diluted to 1×10⁵ per well in 96 well plate. Cells are grown in 96 well plate in 7AG Middlebrook media with 0.1% Tyloxapol. Test drugs are resuspended in 100% DMSO, while kanamycin is resuspended in dH2O. A 2% DMSO solution is added to the highest drug concentration of kanamycin, which is serially diluted down with 2 fold dilutions. The final DMSO concentration in all wells is 1%. Plates are incubated at 37° C. for 4 days. A solution of 1 mg resazurin in 8 mL of PBS is prepared, and 10 μL is added to each well. Plates are read 3 days later, and IC₅₀ and IC₉₀ values are calculated using commercially available software, such as PRISM.

METHOD EXAMPLE. Determination of Minimal Inhibitory Concentrations (MIC). MICs of niclosamide and tribromsalan were measured against Mycobacterium tuberculosis H37Rv (MIB) in axenic log phase culture in vitro, to determine treatment conditions for assessing genome-wide transcriptional effects to treatment. Drugs were prepared as 10 mM stock solutions in dimethyl sulfoxide and stored at −20′C in aliquots, to avoid freeze-thawing. MTB was cultured in Middlebrook 7H9 broth (0.05% Tween 80, 10% ADC) and 96-well microtitre plates containing two-fold dilutions of each compound were inoculated with MTB to a final concentration of 1×10⁵-5×10⁵ CFU/mL. Plates were incubated for 7 days at 37° C. and MIC values then determined by REMA following 16 h incubation with CellTiter-Blue. Wells containing media only were used to correct for background and the fluorescence was measured to determine the MIC, calculated as the concentration of drug that inhibited 90% of growth. Drug-free wells acted as positive controls to screen unrestricted MTB growth and wells free of both drug and bacteria were used as negative controls, to determine the background fluorescence. MIC plates were repeated in biological and technical triplicate, to determine a mean MIC.

METHOD EXAMPLE. Transcriptomics Experimental Details. Log phase MTB was treated with either 10×MIC of test compound for 4 hours and vehicle control MTB treated with DMSO only. Following exposure, MTB RNA was immediately extracted using an established GTC/TRIzol differential lysis method. Samples were extracted in biological triplicate on different days. The resulting mycobacterial RNA was DNase-treated and purified using the RNeasy spin columns (Qiagen). RNA yield and quality were assessed by NanoDrop One (Thermo Scientific, Waltham, MA, USA) and Agilent 2100 Bioanalyzer (Agilent Technologies, Santa Clara, CA, USA). Yields from all samples were similar and as expected, good quality. Mycobacterial ribosomal RNA was then depleted from each RNA sample using RiboCop META kit (Lexogen, Vienna, Austria) resulting in mRNA suitable for RNA-seq. Strand-specific RNA-seq libraries were then prepared using NEBNext Ultra II directional RNA library prep kit (New England Biolabs, Ipswitch, MA, USA) and pooled.

METHOD EXAMPLE. Niclosamide and Tribromsalan were tested against M. tuberculosis, and compared to control kanamycin, as shown in the following table:

| Drug | IC50 (μM) | IC90 (μM) |
|---|---|---|
| Kanamycin | 1.06 | 4.22 |
| Tribromsalan | 67.16 | 76.92 |
| Niclosamide | 1.92 | 2.95 |

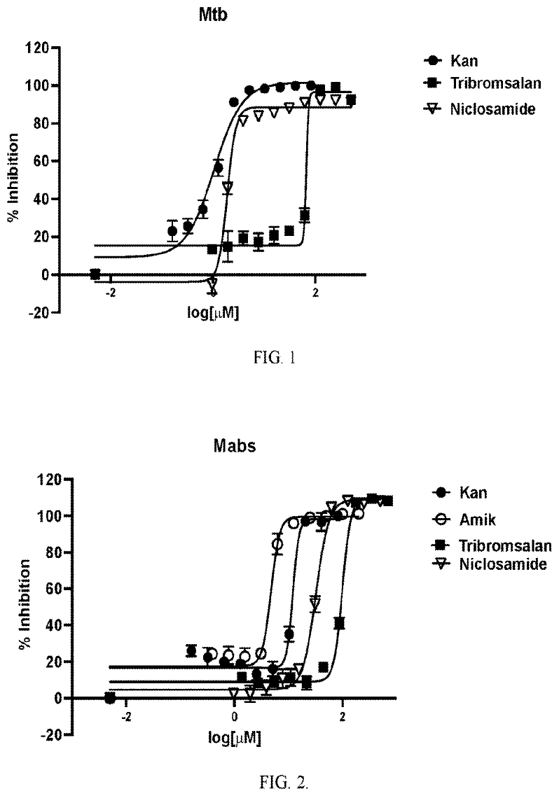

Both niclosamide and tribromsalan showed significant activity against M. tuberculosis, where niclosamide was superior overall to kanamycin. Additional results are shown in FIG. 1.

METHOD EXAMPLE. M. abscessus (ATCC Smooth strain) assay. Frozen stock ATCC Smooth (400 μL) is thawed and inoculated into 5 mL 7AG Middlebrook media with 0.1% Tyloxapol overnight. After 1 day, cells are diluted to to 1×10⁶ CFU/mL (100 μL into wells for 1×10⁵ CFU/well). Cells are grown in the 96 well plate in 7AG Middlebrook media with 0.1% Tyloxapol. Tested drugs are resuspended in 100% DMSO, while kanamycin and amikacin are resuspended in dH2O. A 2% DMSO solution is added to the highest drug concentrations of kanamycin and amikacin, which are serially diluted down with 2 fold dilutions. The final DMSO concentration in all wells is 1%. Plates are incubated at 37° C. for 4 days. A solution of 1 mg resazurin in 8 mL of PBS is prepared, and 20 μL is added to each well. Plates are read after 1 day, and IC₅₀ and IC₉₀ values are calculated using commercially available software, such as PRISM.

METHOD EXAMPLE. Niclosamide and Tribromsalan were tested against M. abscessus, and compared to control kanamycin and amikacin, as shown in the following table:

| Drug | IC50 (μM) | IC90 (μM) |
|---|---|---|
| Kanamycin | 12.11 | 16.07 |
| Amikacin | 4.78 | 7.15 |
| Tribromsalan | 99.75 | 147.4 |
| Niclosamide | 32.23 | 59.1 |

Both niclosamide and tribromsalan showed significant activity against M. abscessus. Additional results are shown in FIG. 2.

The following publications, and each of the additional publications cited herein, are incorporated herein by reference:
[1] WHO. Global Tuberculosis Report 2021. 2021.
[2] Seung K J, Keshavjee S, Rich M L. Multidrug-Resistant Tuberculosis and Extensively Drug-Resistant Tuberculosis. Cold Spring Harb Perspect Med 2015; 5:a017863. doi: 10.1101/cshperspect.a017863.
[3] Arnold A, Cooke G S, Kon O M, Dedicoat M, Lipman M, Loyse A, Chis Ster I, Harrison T S. Adverse Effects and Choice between the Injectable Agents Amikacin and Capreomycin in Multidrug-Resistant Tuberculosis. Antimicrob Agents Chemother 2017; 61 doi: 10.1128/AAC.02586-16.
[4] Anon. Inadequate Treatment.
[5] Zumla A, Chakaya J, Centis R, D'Ambrosio L, Mwaba P, Bates M, Kapata N, Nyirenda T, Chanda D, Mfinanga S, Hoelscher M, Maeurer M, Migliori G B. Tuberculosis treatment and management—an update on treatment regimens, trials, new drugs, and adjunct therapies. Lancet Respir Med 2015:3:220-234. doi: 10.1016/S2213-2600(15)00063-6.
[6] Makarov V, Salina E, Reynolds R C, Kyaw Zin P P, Ekins S. Molecule Property Analyses of Active Compounds for Mycobacterium tuberculosis. J Med Chem 2020; 63:8917-8955. doi: 10.1021/acs.jmedchem.9b02075.
[7] Egorova A. Jackson M. Gavrilyuk V, Makarov V. Pipeline of anti-Mycobacterium abscessus small molecules: Repurposable drugs and promising novel chemical entities. Med Res Rev 2021 doi: 10.1002/med.21798.
[8] Lopeman R C, Harrison J, Desai M, Cox J A G. Mycobacterium abscessus: Environmental Bacterium Turned Clinical Nightmare. Microorganisms 2019; 7 doi: 10.3390/microorganisms7030090.
[9] Ganapathy U S, Dartois V, Dick T. Repositioning rifamycins for Mycobacterium abscessus lung disease. Expert Opin Drug Discov 2019; 14:867-878. doi: 10.1080/17460441.2019.1629414.
[10] Chen B, Ding Y, Wild D J. Assessing drug target association using semantic linked data. PLoS Comput Biol 2012; 8:e1002574. doi: 10.1371/journal.pcbi.1002574.
[11] Lee J A, Shinn P, Jaken S, Oliver S, Willard F S, Heidler S, Peerv R B, Oler J, Chu S, Southall N, Dexheimer T S, Smallwood J, Huang R, Guha R, Jadhav A, Cox K, Austin C P, Simeonov A, Sittampalam G S, Husain S, Franklin N, Wild D J, Yang J J, Sutherland J J, Thomas C J. Novel Phenotypic Outcomes Identified for a Public Collection of Approved Drugs from a Publicly Accessible Panel of Assays. PLoS One 2015; 10:e0130796. doi: 10.1371/joumal.pone.0130796.
[12] Williams A J, Harland L, Groth P, Pettifer S, Chichester C, Willighagen E L, Evelo C T, Blomberg N, Ecker G, Goble C, Mons B. Open PHACTS: semantic interoperability for drug discovery. Drug Discov Today 2012:17:1188-1198. doi: 10.1016/j.drudis.2012.05.016.
[13] Lane T. Russo D P, Zom K M, Clark A M, Korotcov A. Tkachenko V, Reynolds R C, Perryman A L, Freundlich J S, Ekins S. Comparing and Validating Machine Learning Models for Mycobacterium tuberculosis Drug Discovery. Mol Pharm 2018; 15:4346-4360. doi: 10.1021/acs.molpharmaceut.8b00083.
[14] Fan X, Xu J, Files M, Cirillo J D, Endsley J J. Zhou J, Endsley M A. Dual activity of niclosamide to suppress replication of integrated HIV-1 and Mycobacterium tuberculosis (Beijing). Tuberculosis (Edinb) 2019:116S:S28-S33. doi: 10.1016/j.tube.2019.04.008.
[15] Berube B J, Castro L, Russell D, Ovechkina Y, Parish T. Novel Screen to Assess Bactericidal Activity of Compounds Against Non-replicating Mycobacterium abscessus. Front Microbiol 2018; 9:2417. doi: 10.3389/fmicb.2018.02417.
[16] Williams J T, Haiderer E R, Coulson G B, Conner K N, Ellsworth E, Chen C, Alvarez-Cabrera N, Li W, Jackson M, Dick T, Abramovitch R B. Identification of New MmpL3 Inhibitors by Untargeted and Targeted Mutant Screens Defines MmpL3 Domains with Differential Resistance. Antimicrob Agents Chemother 2019:63 doi: 10.1128/AAC.00547-19
[17] Baranyai Z, Kratky M, Vinsova J, Szabo N, Senoner Z, Horvati K, Stolarikova J, David S, Bosze S. Combating highly resistant emerging pathogen Mycobacterium abscessus and Mycobacterium tuberculosis with novel salicylanilide esters and carbamates. Eur J Med Chem 2015; 101:692-704. doi: 10.1016/j.ejmech.2015.07.001.
[18] Tailleux L, Waddell S J, Pelizzola M, Mortellaro A, Withers M, Tanne A, Castagnoli P R, Gicquel B, Stoker N G, Butcher P D, Foti M, Neyrolles O. Probing host pathogen cross-talk by transcriptional profiling of both Mycobacterium tuberculosis and infected human dendritic cells and macrophages. PLoS One 2008:3:e1403. doi: 10.1371/joumal.pone.0001403.

1. A composition comprising a salicylanilide or a prodrug thereof, or a hydrate or solvate of any of the foregoing, or a salt of the foregoing, for use in treating an infection caused at least in part by Mycobacteriaceae.

2. The composition of claim 1 wherein the Mycobacteriaceae include Mycobacterium tuberculosis complex (MTBC), Mycobacterium abscessus complex (MABC), Mycobacterium avium complex (MAC), Mycobacteria causing leprosy, Mycobacteria comorbid with cystic fibrosis, or M. ulcerans, or any combination of the foregoing.

3. The composition of claim 1 wherein the Mycobacteriaceae include Mycobacterium tuberculosis complex (MTBC).

4. The composition of claim 1 wherein the Mycobacteriaceae include Mycobacterium abscessus complex (MABC).

5. The composition of claim 1 wherein the Mycobacteriaceae include Mycobacterium avium complex (MAC).

6. The composition of claim 1 wherein the Mycobacteriaceae include Mycobacteria causing leprosy.

7. The composition of claim 1 wherein the Mycobacteriaceae include Mycobacteria comorbid with cystic fibrosis, such as M. chitae or M. fallax, or a combination thereof.

8. The composition of claim 1 wherein the composition is configured for oral administration.

9. The composition of claim 1 wherein the composition is configured for administration by inhalation.

10. The composition of claim 1 wherein the host animal is a mammal, such as livestock, a companion animal, or a human.

11. The composition of claim 1 wherein the host animal is immunocompromised.

12. The composition of claim 1 wherein the salicylanilide is selected from the formula 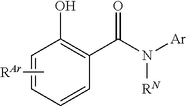
and prodrugs thereof; and salts of any of the foregoing; where
R^{Ar} represents one or more aryl substituents;
R^{N} is H, alkyl, or acyl; and
Ar is an optionally substituted aryl group or heteroaryl group.

13. The composition of claim 12 wherein R^{Ar}r is one or more halo groups, hydroxy, amino, or heteroaryl, or any combination of the foregoing.

14. The composition of claim 1 wherein Ar is phenyl or substituted phenyl.

15. The composition of claim 12 wherein R^{N} is H.

16. The composition of claim 12 wherein the prodrug is an 2-O-acyl derivative.

17. The composition of claim 12 wherein the compound is selected from the formulae 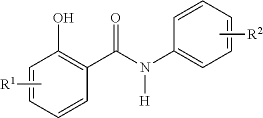
where R¹ is 5-Cl and R² is 4-Cl, R¹ is 5-Cl and R² is c-Cl, or R¹ is 5-Cl and R² is 4-Br.

18. The composition of any one of claims 1 to 11 wherein the composition comprises salicylanilide, niclosamide, oxyclozanide, rafoxanide, closantel, 5-chlorosalicylanilide (CSA), 4′-bromosalicylanilide (BSA), 5-bromo-4′-chlorosalicylanilide (BCSA), 3,3′4′,5-tetrachlorosalicylanilide (TCSA), bromochlorosalicylanilide, 5-bromo-N-(3,5-dichlorophenyl)-2-hydroxy-benzamide, 5-chloro-N-(2,4-difluorophenyl)-2-hydroxy-benzamide, 3,5-dichloro-N-(2,4-difluorophenyl)-4-pyrrol-1-yl-benzamide, 4-amino-3,5-dichloro-N-(3,5-dichlorophenyl)benzamide, 5-chloro-N-(2-chloro-4-nitro-phenyl)-2-hydroxy-benzamide, clioxanide, or any combination thereof.

19. The composition of claim 1 wherein the composition comprises dibromsalan (DBS), metabromsalan (MBS), tribromsalan (TBS, 3,5,4′-tribromosalicylanilide), resorantel, 4′,5-dibromosalicylanilide or a combination thereof.

20. (canceled)

21. A method for treating a Mycobacteriaceae infection in a host animal, the method comprising administering a therapeutically effective amount of the composition of claim 1, or a unit dose comprising the composition of claim 1, in single or divided form, to the host animal.

22. (canceled)
